(19) United States
(12) Patent Application Publication
(10) Pub. No.: US 2021/0015680 A1
(43) Pub. Date: Jan. 21, 2021

(54) THERAPEUTIC ELASTIC BANDAGE FOR MODULATING THE ENDOCANNABINOID SYSTEM

(71) Applicants: Steven Robert ERBST, Los Gatos, CA (US); Cameron Patrick ERBST, Los Gatos, CA (US); Mathew Lee WEBER, Portland, OR (US)

(72) Inventors: Steven Robert ERBST, Los Gatos, CA (US); Cameron Patrick ERBST, Los Gatos, CA (US); Mathew Lee WEBER, Portland, OR (US)

(21) Appl. No.: 17/062,379

(22) Filed: Oct. 2, 2020

Related U.S. Application Data
(63) Continuation of application No. PCT/US2018/063516, filed on Dec. 1, 2018.
(60) Provisional application No. 62/660,243, filed on Apr. 19, 2018.
(60) Provisional application No. 62/653,240, filed on Apr. 5, 2018.

(30) Foreign Application Priority Data
Dec. 1, 2018 (US) PCT/US2018/063516

(51) Int. Cl. A61F 13/02; A61K 31/05; A61K 31/351; C09J 7/35; C09J 7/38; A61K 9/70

(52) U.S. Cl. CPC A61F 13/025; A61K 31/05; A61K 31/351; A61F 13/0256; A61K 45/06; C09J 7/385; A61K 9/7061; A61K 9/7084; C09J 7/35

(57) ABSTRACT

The invention provides articles of manufacture, compositions, and methods related to a therapeutic elastic bandage such as a kinesiology tape comprising a medicament to modulate the endocannabinoid system of a subject.

### CROSS-REFERENCE TO RELATED APPLICATIONS

This application is a continuation of International Patent Application No. PCT/US2018/063516, filed Dec. 1, 2018, which claims the priority benefit of U.S. Provisional Application Ser. No. 62/653,240, filed Apr. 5, 2018, and of U.S. Provisional Application Ser. No. 62/660,243, filed Apr. 19, 2018, each of which are hereby incorporated by reference in their entirety.

### BACKGROUND OF THE INVENTION

A wide variety of bandages are known in the art. For example, traditional elastic bandages are used to prevent and treat injuries such as bruises and sprains through local compression, thereby providing support and helping to reduce swelling and inflammation. Kinesiology tape was developed as a more advanced form of elastic bandage having an adhesive on one side for attachment to skin. Kinesiology tape is applied to create a pulling force on skin, thereby promoting relief from pain, better blood flow to an injured area, and enhanced lymphatic drainage to reduce swelling and inflammation. Improvements to such elastic bandages have related to, for example, the specific materials used, the degree of elasticity of the materials, and the adhesive applied to attach kinesiology tape to a user's body.

Both traditional elastic bandages and more advanced kinesiology tapes do not work well to prevent and/or treat certain conditions experienced by some individual users. Accordingly, there is a need for improvements to currently available elastic bandages and kinesiology tapes.

### SUMMARY OF THE INVENTION

In one embodiment, the invention provides a therapeutic bandage comprising an elastic material and an additive, wherein the additive comprises a medicament, and wherein the medicament comprises a modulator of the endocannabinoid system. In some embodiments, the bandage is capable of delivering a biologically effective dose of the medicament to at least a portion of a body of a subject. In some embodiments, the material comprises a front surface and a back surface, and wherein an adhesive material is applied to at least one surface. In some embodiments, the material comprises a kinesiology tape. In some embodiments, the medicament does not have a psychoactive effect. In some embodiments, the medicament comprises a cannabinoid. In some embodiments, the cannabinoid is natural. In some embodiments, the cannabinoid is synthetic. In some embodiments, the medicament comprises tetrahydrocannabinol (THC). In some embodiments, the THC comprises delta-9-THC-acid and/or delta-8-THC. In some embodiments, the medicament comprises cannabidiol (CBD). In some embodiments, the CBD comprises CBD-acid. In some embodiments, the medicament comprises cannabichromene (CBC). In some embodiments, the CBC comprises CBC-acid. In some embodiments, the medicament comprises cannabigerol (CBG). In some embodiments, the CBG comprises CBG-acid. In some embodiments, the medicament comprises only one type of cannabinoid. In some embodiments, the cannabinoid is CBD. In some embodiments, the medicament comprises at least two different cannabinoids. In some embodiments, the at least two different cannabinoids comprise CBD and THC. In some embodiments, the medicament has a CBD/THC ratio of about 1:1 by weight. In some embodiments, the medicament comprises a fatty acid amide hydrolase (FAAH) inhibitor.

In one embodiment, the invention provides a therapeutic bandage comprising an elastic material and an additive, wherein the additive comprises a medicament, and wherein the medicament comprises a modulator of the endocannabinoid system. In some embodiments, the additive comprises a fatty acid amide hydrolase (FAAH) inhibitor. In some embodiments, the additive comprises a terpene. In some embodiments, the additive comprises one or more terpene selected from the group consisting of eucalyptol, borneol, alpha-bisabolo, camphene, delta-3-carene, humulene, linalool, beta-caryophyllene, myrcene (or beta-myrcene), alpha-pinene, and limonene. In some embodiments, the additive comprises an essential oil. In some embodiments, the additive comprises one or more essential oil selected from the group consisting of chamomile, lavender, eucalyptus, peppermint, rosemary, clary sage, juniper, yarrow, clove, wintergreen, cayenne, ginger, sandalwood, marjoram, helichrysum, frankincence, vetiver, black pepper, lemongrass, rose geranium, bergamot, chenopodium, ylang-ylang, and L-menthol. In some embodiments, the additive comprises a permeation enhancer. In some embodiments, the permeation enhancer comprises a surfactant. In some embodiments, the surfactant is selected from the group consisting of sodium lauryl sulfate, polyoxyethylene-2-Oleyl ether, and polyoxyethylene-2-stearyl ether. In some embodiments, the permeation enhancer comprises an oil. In some embodiments, the oil is selected from the group consisting of medium chain triglycerides (MCT) oil, coconut oil, and palm kernel oil. In some embodiments, the permeation enhancer comprises a fatty acid. In some embodiments, the fatty acid is selected from the group consisting of a triglyceride, one or more medium-chain triglycerides (MCTs), oleic acid, lauric acid, myristic acid, and capric acid. In some embodiments, the permeation enhancer comprises a glycol. In some embodiments, the glycol is selected from the group consisting of diethylene glycol and tetraethylene glycol. In some embodiments, the permeation enhancer comprises a compound with an alkyl group. In some embodiments, the permeation enhancer comprises a compound with an aryl group. In some embodiments, the permeation enhancer comprises an azone. In some embodiments, the additive comprises one or more permeation enhancer selected from the group consisting of ethanol, chenopodium, ylang-ylang, L-menthol, eucalyptus, sodium lauryl sulfate, polyoxyetylene-2-oleyl ether, polyoxyethylene-2-stearyl ether, medium chain triglycerides (MCT) oil, coconut oil, palm kernel oil, a triglyceride, one or more medium-chain triglycerides (MCTs), oleic acid, lauric acid, myristic acid, capric acid, diethylene glycol, tetraethylene glycol, and cyclic urea.

In one embodiment, the additive and/or medicament is formulated with or in a carrier for enhanced delivery to a user and the carrier is a vesicular carrier. In some embodiments, the vesicular carrier is selected from the group consisting of ethosomes, emulsomes, liposomes, enzymosomes, virosomes, pharmacosomes, transferosomes, sphingosomes, and niosomes. In some embodiments, the additive comprises a formulation comprising a terpene, an essential oil, and a cannabinoid. In some embodiments, the additive comprises a formulation comprising an ethosomal carrier, CBD and THC at a ratio of about 1:1 CBD:THC, limonene, and beta-myrcene. In some embodiments, the additive comprises a formulation comprising an ethosomal carrier, a cannabinoid comprising CBD only, limonene, and beta-myrcene. In some embodiments, the additive comprises a formulation comprising MCT oil, CBD and THC at a ratio of about 1:1 CBD:THC, limonene, and beta-myrcene. In some embodiments, the additive comprises a formulation comprising MCT oil, a cannabinoid comprising CBD only, limonene, and beta-myrcene. In some embodiments, the additive comprises a formulation comprising a vesicular carrier, CBD and THC at a ratio of about 10:1-1:10 CBD:THC, and a terpene.

In one embodiment, the invention provides a therapeutic bandage comprising an elastic material and an additive, wherein the additive comprises a medicament, and wherein the medicament comprises a modulator of the endocannabinoid system. In some embodiments, the material is comprised of a single elastic layer. In some embodiments, the material comprises multiple elastic layers. In some embodiments, the material comprises microneedles. In some embodiments, the material is capable of dermabrasion. In some embodiments, the material is water-resistant. In some embodiments, the material comprises synthetic material. In some embodiments, the synthetic material is selected from the group consisting of nylon, Lycra®, Spandex®, elastane, or non-woven polyurethane. In some embodiments, the material comprises natural material. In some embodiments, the natural material comprises cotton. In some embodiments, the material comprises an adhesive material. In some embodiments, the adhesive is stretch-activated and/or heat-activated. In some embodiments, the adhesive is hypo-allergenic. In some embodiments, the adhesive is latex-free. In some embodiments, the adhesive comprises an acrylic or poly-acrylic adhesive. In some embodiments, the adhesive comprises a StrataGel®.

In one embodiment, the invention provides a therapeutic bandage comprising an elastic material and an additive, wherein the additive comprises a medicament, and wherein the medicament comprises a modulator of the endocannabinoid system. In some embodiments, the bandage comprises a built-in reservoir. In some embodiments, the built-in reservoir comprises the additive. In some embodiments, the additive is infused into a portion of one or more layer of material.

In one embodiment, the invention provides a method for delivering an effective dose of a modulator of the endocannabinoid system to a portion of a body of a subject, comprising attaching or applying a therapeutic bandage to the skin of the subject, wherein the therapeutic bandage comprises an elastic material and an additive, wherein the additive comprises a medicament, and wherein the medicament comprises the modulator of the endocannabinoid system. In some embodiments, the method promotes lymphatic drainage, improves muscle tone, provides pain relief, reduces swelling, reduces inflammation, reduces muscle spasms, reduces muscle cramps, reduces muscle fatigue, promotes recovery from bruises, promotes recovery from contusions, alleviates nausea, promotes muscle relaxation, provides muscle support, provides joint support, promotes muscle recovery, prevents muscle spasms/cramping, relieves muscle spasms/cramping, promotes muscle recovery, supports injured muscle without restricting range of movement, enhances strength and muscle tone, relieves pressure on pain receptors directly under the skin, facilitates blood circulation, facilitates lymphatic circulation, facilitates lymphatic drainage, drains swelling, facilitates removal of inflammatory cells away from an injured area quickly, relieves lymphedema, limits buildup of lactic acid, facilitates removal of lactic acid, delays onset of muscle soreness, and/or ameliorates post-exercise muscle soreness. In some embodiments, the bandage is attached/applied to the skin of the subject for at least about 1 hour, at least about 3 hours, at least about 6 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 18 days, at least about 22 days, at least about 26 days, or at least about 30 days. In some embodiments, the medicament comprises a cannabinoid. In some embodiments, the subject is a human. In some embodiments, the subject is an animal.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, including the accompanying drawings.

FIG. 1 shows an embodiment of a kinesiology tape comprising a single layer of elastic material, an adhesive layer that does not fully cover the skin-facing surface of the tape, and an additive comprising a medicament to modulate the endocannabinoid system of a subject.

FIG. 2 shows an embodiment of a kinesiology tape comprising a single layer of elastic material, an adhesive layer that fully covers the skin-facing surface of the tape, and a removable protective sheet on the adhesive layer.

FIG. 3 shows an embodiment of a kinesiology tape comprising multiple layers of elastic material and an adhesive layer that does not fully cover the skin-facing surface of the tape.

FIG. 4 shows an embodiment of a kinesiology tape comprising multiple layers of elastic material and an adhesive layer that fully covers the skin-facing surface of the tape.

FIG. 5 shows an embodiment of a kinesiology tape comprising multiple layers of elastic material, an adhesive layer that does not fully cover the skin-facing surface of the tape, a reservoir between the multiple layers, a different layer of elastic material under the reservoir, and an additive comprising a medicament to modulate the endocannabinoid system of a subject.

FIG. 6 shows an embodiment of a kinesiology tape comprising multiple layers of elastic material, an adhesive layer that fully covers the skin-facing surface of the tape, a reservoir between the multiple layers, and an additive comprising a medicament to modulate the endocannabinoid system of a subject.

FIG. 7 shows an embodiment of a kinesiology tape comprising multiple layers of elastic material, multiple reservoirs between the multiple layers, an adhesive layer that does not fully cover the skin-facing surface of the tape, and an additive comprising a medicament to modulate the endocannabinoid system of a subject.

FIG. 8 shows an embodiment of a kinesiology tape, wherein the locations of additive and adhesive material do not overlap.

FIG. 9 shows an embodiment of a kinesiology tape, wherein the locations of additive and adhesive material overlap.

FIG. 10 shows an embodiment of a kinesiology tape, wherein the locations of reservoirs and adhesive material do not overlap.

FIG. 11 shows an embodiment of a kinesiology tape, wherein the locations of reservoirs and adhesive material overlap.

FIG. 12 shows an embodiment of a therapeutic elastic bandage on the arm of a subject.

FIG. 13 shows an embodiment of a kinesiology tape applied to the skin by the lower back of a subject.

FIG. 14 shows an embodiment of a kinesiology tape applied to the skin by the knee of a subject.

FIG. 15 shows an embodiment of a kinesiology tape applied to the skin by the elbow of a subject.

### DETAILED DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention. It is intended that the following claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

As used in the specification and claims, the singular forms “a”, “an” and “the” include plural references unless the context clearly dictates otherwise.

The term “bandage”, as used herein, refers to a strip of flexible material used to bind a wound, to protect a body part from injury, to protect an injured body part, to heal an injured body part, or to provide support to or to restrict the movement of a body part.

The term “kinesiology tape”, as used herein, refers to an elastic and flexible material having an adhesive at least on one side so that the kinesiology tape can be attached to the skin of a subject. For example, kinesiology tape may be stretchable to a total stretched dimension of at least 5%, 10%, 20%, 30%, 40%, 50%, 60%, 70%, 80%, 90%, 100%, 110%, 120%, 130%, 140%, 150%, 160%, 170%, 180%, 190%, or 200% of its unstretched dimension in the direction of the material's width and/or length. In some cases, the kinesiology tape may be applied to the skin of a subject to treat an injury or ailment, for example an athletic injury. In other cases, the kinesiology tape may be applied to the skin of a subject to prevent an injury or ailment. The kinesiology tape may also be used to provide support to or to restrict the movement of a body part.

The term “modulator”, as used herein, refers to an activator, an inhibitor, or both. To “modulate”, as used herein, refers to act on (for example, a receptor, a pathway, or a system) as an activator, an inhibitor, or both.

The terms “endocannabinoid system” or “endogenous cannabinoid system” or “ECS”, as used herein, generally refer to a biological system comprised of cannabinoid receptors, endogenous cannabinoids (endocannabinoids), and the enzymes responsible for the synthesis and degradation of the endocannabinoids. A modulator may influence any one or more component(s) of a subject's ECS. The ECS is present in humans and other animals.

The terms “elastic” or “stretch”, and their derivatives, as used herein, refer to the property of a material to be made or to be capable of being made longer or wider without tearing or breaking upon application of a force. As used herein, an elastic material that is stretched will return to at least a portion of its original length and/or width upon removal of the force that caused it to stretch.

The invention provides articles of manufacture, compositions, and methods related to a therapeutic elastic bandage and/or kinesiology tape comprising a medicament to modulate the endocannabinoid system of a subject. In one embodiment, the invention provides a therapeutic bandage comprising an elastic material and an additive, wherein the additive comprises a medicament, and wherein the medicament comprises a modulator of the endocannabinoid system. In another embodiment, the invention provides a therapeutic bandage comprising an elastic material and an additive, wherein the additive comprises a medicament, wherein the medicament comprises a modulator of the endocannabinoid system, and wherein the elastic material comprises a kinesiology tape. The elastic material of the therapeutic elastic bandage and/or kinesiology tape may have an outer surface (i.e., the side facing away from the skin when the therapeutic elastic bandage and/or kinesiology tape is applied or attached to a subject) and an inner surface (i.e., the side facing the skin when the therapeutic elastic bandage and/or kinesiology tape is applied or attached to a subject), such that the medicament is capable of diffusing through or via the inner surface to a portion of a body of a subject using the elastic bandage and/or kinesiology tape.

In some embodiments, the therapeutic elastic bandage and/or kinesiology tape is water-resistant. In some embodiments, the therapeutic elastic bandage and/or kinesiology tape is waterproof. In some embodiments, the therapeutic elastic bandage and/or kinesiology tape is breathable. In some embodiments, the therapeutic elastic bandage and/or kinesiology tape is waterproof and breathable. In some embodiments, the therapeutic elastic bandage and/or kinesiology tape is hypoallergenic. In some embodiments, the therapeutic elastic bandage and/or kinesiology tape is latex-free. In some embodiments, the therapeutic elastic bandage and/or kinesiology tape is zinc-free.

The elastic material of the therapeutic elastic bandage and/or kinesiology tape may be produced in any dimensions or sizes appropriate for application or attachment to a subject's body. The subject may be a human subject or an animal subject. For example, the elastic material may be produced in pediatric through adult sizes and in different sizes and shapes for application or attachment to different body regions. In some embodiments, the elastic material is substantially flat. In some embodiments, as applied or attached to a subject, the elastic material preferably conforms to the subject's body. In some embodiments, the kinesiology tape is formed from a strip of material having a length that is substantially longer than the dimension of its width, and a height that is substantially shorter than the dimension of its width and length. In some embodiments, the elastic material of the kinesiology tape has a length that is two-times or more, three-times or more, four-times or more, five-times or more, six-times or more, eight-times or more, ten-times or more, fifteen-times or more, or twenty-times or more the dimension of its width.

The elastic material of the therapeutic elastic bandage and/or kinesiology tape is preferably stretchable at least some percent of its unstretched dimension at least in the material's width and/or length. In some embodiments, the elastic material of the therapeutic elastic bandage and/or kinesiology tape is stretchable to a total stretched dimension of at least 5%, 10%, 20%, 30%, 40%, 50%, 60%, 70%, 80%, 90%, 100%, 110%, 120%, 130%, 140%, 150%, 160%, 170%, 180%, 190%, or 200% of its unstretched dimension in the direction of the material's width and/or length. In some embodiments, the kinesiology tape is more stretchable in one dimension than in another dimension. In some embodiments, the kinesiology tape is more stretchable in the direction of the tape's length than in the direction of the tape's width. In some embodiments, the kinesiology tape is stretchable to a total stretched dimension of at least 5%, 10%, 20%, 30%, 40%, 50%, 60%, 70%, 80%, 90%, 100%, 110%, 120%, or 130% of its unstretched dimension in the direction of the tape's width. In some embodiments, the kinesiology tape is stretchable to a total stretched dimension of at least 5%, 10%, 20%, 30%, 40%, 50%, 60%, 70%, 80%, 90%, 100%, 110%, 120%, 130%, 140%, 150%, 160%, 170%, 180%, 190%, or 200% of its unstretched dimension in the direction of the tape's length. In some embodiments, the kinesiology tape is stretchable to a total stretched dimension of no more than 5%, 10%, 20%, 30%, 40%, 50%, 60%, 70%, 80%, 90%, 100%, 110%, 120%, or 130% of its unstretched dimension in a first dimension and is stretchable at least 5%, 10%, 20%, 30%, 40%, 50%, 60%, 70%, 80%, 90%, 100%, 110%, 120%, 130%, 140%, 150%, 160%, 170%, 180%, 190%, or 200% of its unstretched dimension in a second dimension orthogonal to the first dimension. In some embodiments, the kinesiology tape is stretchable to a total stretched dimension of no more than 5%, 10%, 20%, 30%, 40%, 50%, 60%, 70%, 80%, 90%, 100%, 110%, 120%, or 130% of its unstretched dimension in the direction of the tape's width and is stretchable at least 5%, 10%, 20%, 30%, 40%, 50%, 60%, 70%, 80%, 90%, 100%, 110%, 120%, 130%, 140%, 150%, 160%, 170%, 180%, 190%, or 200% of its unstretched dimension in the direction of the tape's length. In some embodiments, the kinesiology tape is stretchable to a total stretched dimension of at least 30% of its unstretched dimension in the direction of the tape's length. In some embodiments, the kinesiology tape is stretchable to a total stretched dimension of at least 130% of its unstretched dimension in the direction of the tape's length. In some embodiments, the kinesiology tape is stretchable to a total stretched dimension of at least 50% of its unstretched dimension in the direction of the tape's length. In some embodiments, the kinesiology tape is stretchable to a total stretched dimension of at least 20% to 40% of its unstretched dimension in the direction of the tape's length. In some embodiments, the kinesiology tape is stretchable to a total stretched dimension of at least 120% to 140% of its unstretched dimension in the direction of the tape's length. In some embodiments, the kinesiology tape is stretchable to a total stretched dimension of at least 150% of its unstretched dimension in the direction of the tape's length. In some embodiments, the kinesiology tape is stretchable to a total stretched dimension of at least 80% of its unstretched dimension in the direction of the tape's length. In some embodiments, the kinesiology tape is stretchable to a total stretched dimension of at least 180% of its unstretched dimension in the direction of the tape's length. In some embodiments, the kinesiology tape is stretchable to a total stretched dimension of no more than 110% of its unstretched dimension in the direction of the tape's width and is stretchable to a total stretched dimension of at least 130% of its unstretched dimension in the direction of the tape's length. In some embodiments, the kinesiology tape is stretchable to a total stretched dimension of no more than 110% of its unstretched dimension in the direction of the tape's width and is stretchable to a total stretched dimension of at least 150% of its unstretched dimension in the direction of the tape's length. In some embodiments, the kinesiology tape is stretchable to a total stretched dimension of no more than 110% of its unstretched dimension in the direction of the tape's width and is stretchable to a total stretched dimension of at least 180% of its unstretched dimension in the direction of the tape's length.

The elastic material of the therapeutic elastic bandage and/or kinesiology tape may be comprised of one or more layers of natural and/or artificial (i.e., synthetic) material. In some cases, the elastic material comprises an elastic woven fabric. The elastic material may be breathable. In some embodiments, the elastic material is water-resistant. In some embodiments, the elastic material is waterproof. In some embodiments, the elastic material comprises an antibacterial material. In some embodiments, the elastic material comprises a porous layer or membrane. In some embodiments, the elastic material comprises a natural material, for example cotton. The elastic material may comprise one or more different natural materials. In some embodiments, the elastic material comprises a synthetic material, for example nylon, Lycra®, Spandex®, elastane, or non-woven polyurethane. The elastic material may comprise one or more different synthetic materials. In some embodiments, the elastic material is made of a combination of natural and synthetic materials. The elastic material may be similar or identical to the material used to construct a brand of kinesiology tape, for example, RockTape®, Kinesio Tape®, MuscleAidTape®, KT Tape®, K-Active®, SpiderTech®, PerformTex®, KinesioTex®, or Physix Gear Sport®. In some embodiments, the elastic material comprises ACE (“All Cotton Elastic”) material. In some embodiments, the elastic material comprises about or exactly 100% cotton fabric. In some embodiments, the elastic material comprises about or exactly 100% synthetic fabric. In some embodiments, the elastic material comprises about 97% cotton and about 3% Nylon 6/12. In some embodiments, the elastic material comprises about 98% cotton and about 2% Spandex. In some embodiments, the elastic material comprises about 95% cotton and about 5% Spandex. In some embodiments, the elastic material comprises about 93% cotton and about 7% Elastane. In some embodiments, the elastic material comprises between about 80%-97% cotton, wherein the remainder non-cotton portion of the elastic material is selected from nylon, Lycra®, Spandex®, elastane, or non-woven polyurethane, or any combination thereof.

The elastic material of the therapeutic elastic bandage and/or kinesiology tape may comprise microneedles on a surface that is facing or in contact with a user's skin. In some embodiments, the elastic material is capable of dermabrasion. In some embodiments, the elastic material is capable of sensorimotor stimulation.

The elastic material of the therapeutic elastic bandage and/or kinesiology tape may comprise an adhesive material. For example, multiple layers of the elastic material may be bound by an adhesive. Any suitable adhesive for binding layers of elastic fabric may be used. In some embodiments, the elastic material does not comprise an adhesive material on a surface that is in contact with a user's skin. In some embodiments, the elastic material comprises an adhesive material on a surface that is in contact with the skin of a user (e.g., kinesiology tape), thereby allowing attachment of the elastic material to the user's skin. Any suitable adhesive for binding stretched woven fabric to the skin of a user may be used. In some embodiments, the elastic material comprises an adhesive material on a surface that is in contact with a user's skin and the adhesive is applied to the surface in any manner to allow for attachment of the elastic material to a user's skin. For example, the adhesive may be applied as a thin layer throughout the surface of the tape that will be attached to the skin of a user. In some embodiments, the elastic material comprises an adhesive material on a surface that is in contact with a user's skin and the adhesive is applied to the surface in a specific pattern. For example, the adhesive may be applied in a wave pattern. In some embodiments, the elastic material comprises an adhesive material on a surface that is in contact with a user's skin and the adhesive is applied to the surface in a substantially random pattern. In some embodiments, the adhesive used to attach the tape to the skin of a user is a pressure-sensitive adhesive. In some embodiments, the adhesive used to attach the tape to the skin of a user is activated by stretching the tape beyond its original dimension(s). In some embodiments, the adhesive used to attach the tape to the skin of a user is a heat-activated adhesive. In some embodiments, the adhesive used to attach the tape to the skin of a user is an adhesive made to medical standards. In some embodiments, the adhesive used to attach the tape to the skin of a user is a hypoallergenic adhesive. In some embodiments, the adhesive used to attach the tape to the skin of a user is a latex-free adhesive. In some embodiments, the adhesive used to attach the tape to the skin of a user is a water-resistant adhesive. In some embodiments, the adhesive used to attach the tape to the skin of a user is a waterproof adhesive. In some embodiments, the adhesive used to attach the tape to the skin of a user is a sweat-resistant adhesive. In some embodiments, the adhesive used to attach the tape to the skin of a user is a latex-free adhesive. In some embodiments, the adhesive used to attach the tape to the skin of a user is an anti-inflammatory adhesive. In some embodiments, the adhesive used to attach the tape to the skin of a user is a zinc-free adhesive. In some embodiments, the adhesive used to attach the tape to the skin of a user allows for wearing of the tape at least for several hours. In some embodiments, the adhesive used to attach the tape to the skin of a user allows for wearing of the tape at least for one or more days. In some embodiments, the adhesive used to attach the tape to the skin of a user allows for wearing of the tape at least for several days. In some embodiments, the adhesive used to attach the tape to the skin of a user allows for wearing of the tape at least for about 2-3 days. In some embodiments, the adhesive used to attach the tape to the skin of a user allows for wearing of the tape at least for about 3-5 days. In some embodiments, the adhesive used to attach the tape to the skin of a user allows for wearing of the tape at least for about 7 days. In some embodiments, the adhesive used to attach the tape to the skin of a user allows for wearing of the tape at least for about 14 days. In some embodiments, the adhesive used to attach the tape to the skin of a user allows for wearing of the tape at least for about 30 days. In some embodiments, the adhesive used to attach the tape to the skin of a user comprises an acrylic or poly-acrylic adhesive. In some embodiments, the adhesive used to attach the tape to the skin of a user comprises a gel adhesive. In some embodiments, the adhesive used to attach the tape to the skin of a user comprises a StrataGel®.

The elastic material of the therapeutic elastic bandage and/or kinesiology tape may comprise an additive. Any one or more ingredient of the additive may be adjusted to the needs of an individual user, for example to maximize efficacy and minimize side effects. In some embodiments, the elastic material may concentrate, be infused with, integrated with, or otherwise include the additive. For example, the additive may be infused into one or more layer of the elastic material. In embodiments comprising multiple layers of elastic material, a reservoir formed between layers of the elastic material may comprise the additive. In some embodiments, the elastic material comprises a porous (or semi-permeable) layer or membrane between a reservoir formed between layers of the elastic material and an adhesive layer. In other embodiments, the elastic material comprises a porous (or semi-permeable) layer or membrane between a reservoir formed between layers of the elastic material and a portion of a user's skin. In yet other cases, the adhesive material/layer of a kinesiology tape may comprise the additive. In some embodiments, the additive comprises only natural component(s). In some embodiments, the additive comprises only synthetic component(s). In some embodiments, the additive comprises natural and synthetic components. In some embodiments, the additive may be in direct contact with the skin of a user. In some embodiments, the additive may be in indirect contact with the skin of a user. The additive may be in liquid, gel, powder, capsule, crystalline, or other forms for absorption into and through the tissues and organs of the body of a user of the therapeutic elastic bandage and/or kinesiology tape.

The additive may comprise a medicament. The concentration of a medicament used in the additive may be adjusted to an individual user's needs, for example to maximize efficacy and minimize side effects. In some embodiments, the medicament comprises a modulator of the endocannabinoid system. In some embodiments, the medicament comprises only natural component(s). In some embodiments, the medicament comprises only synthetic component(s). In some embodiments, the medicament comprises natural and synthetic components. In some embodiments, the medicament does not have or produce a psychoactive effect in a user of the therapeutic elastic bandage and/or kinesiology tape. In some embodiments, the medicament comprises a fatty acid amide hydrolase (FAAH) inhibitor. In some embodiments, the medicament comprises a cannabinoid. The cannabinoid may be natural. The cannabinoid may be synthetic. In some embodiments, the medicament comprises tetrahydrocannabinol (THC). In some embodiments, the medicament comprises delta-9-THC-acid. In some embodiments, the medicament comprises delta-8-THC. In some embodiments, the medicament comprises delta-9-THC-acid and delta-8-THC. In some embodiments, the medicament comprises cannabidiol (CBD). In some embodiments, the medicament comprises CBD-acid. In some embodiments, the medicament comprises cannabichromene (CBC). In some embodiments, the medicament comprises CBC-acid. In some embodiments, the medicament comprises cannabigerol (CBG). In some embodiments, the medicament comprises CBG-acid. In some embodiments, the medicament comprises cannabigerivarin (CBGV). In some embodiments, the medicament comprises CBGV-acid (CBGVA or cannabigerovarinic acid). In some embodiments, the medicament comprises tetrahydrocannabivarin (THCV). In some embodiments, the medicament comprises THCV-acid (THCVA or tetrahydrocanabivarinic acid). In some embodiments, the medicament comprises cannabidivarin (CBDV). In some embodiments, the medicament comprises CBDV-acid (CBDVA or cannabidivarinic acid). In some embodiments, the medicament comprises cannabichromevarin (CBCV). In some embodiments, the medicament comprises CBCV-acid (CBCVA or cannabichromevarinic acid). In some embodiments, the medicament comprises cannabinol (CBN). In some embodiments, the medicament comprises only one type of cannabinoid. In some embodiments, the medicament comprises only one type of cannabinoid, wherein the only one type of cannabinoid is CBD. In some embodiments, the medicament comprises at least two different cannabinoids (e.g., a first cannabinoid and a second cannabinoid). In some embodiments, the medicament comprises a first cannabinoid and a second cannabinoid, wherein the ratio of the first cannabinoid to the second cannabinoid in the medicament is about 0.1:1.0, 0.2:1.0, 0.3:1.0, 0.4:1.0, 0.5:1.0, 0.6:1.0, 0.7:1.0, 0.8:1.0, 0.9:1.0, 1.0:1.0, 1.0:0.9, 1.0:0.8, 1.0:0.7, 1.0:0.6, 1.0:0.5, 1.0:0.4, 1.0:0.3, 1.0:0.2, or 1.0:0.1 by weight. In some embodiments, the medicament comprises CBD and THC. In some embodiments, the medicament comprises CBD and THC at a CBD/THC ratio of about 1:1 by weight. In some embodiments, the medicament comprises CBD and THC at a CBD/THC ratio of about 0.1:1.0, 0.2:1.0, 0.3:1.0, 0.4:1.0, 0.5:1.0, 0.6:1.0, 0.7:1.0, 0.8:1.0, 0.9:1.0, 1.0:1.0, 1.0:0.9, 1.0:0.8, 1.0:0.7, 1.0:0.6, 1.0:0.5, 1.0:0.4, 1.0:0.3, 1.0:0.2, or 1.0:0.1 by weight. In some embodiments, the medicament comprises CBD and THC at a CBD/THC ratio of about 0.50:1.00, 0.55:1.00, 0.60:1.00, 0.65:1.00, 0.70:1.00, 0.75:1.00, 0.80:1.00, 0.85:1.00, 0.90:1.00, 0.95:1.00, 1.00:1.00, 1.05:1.00, 1.10:1.00, 1.15:1.00, 1.20:1.00, 1.25:1.00, 1.30:1.00, 1.35:1.00, 1.40:1.00, 1.45:1.00, 1.50:1.00, 1.55:1.00, 1.60:1.00, 1.65:1.00, 1.70:1.00, 1.75:1.00, 1.80:1.00, 1.85:1.00, 1.90:1.00, 1.95:1.00, or 2.00:1.00.

The additive may comprise a terpene, a terpenoid and/or a flavonoid. In some cases, a single type of terpene, terpenoid, or flavonoid is used. In other cases, a mixture of different terpenes, terpenoids, and/or flavonoids is used. In some embodiments, one or more terpene is selected from eucalyptol, borneol, alpha-bisabolo, camphene, delta-3-carene, humulene, linalool, beta-caryophyllene, myrcene (or beta-myrcene), alpha-pinene, or limonene.

The additive may comprise an essential oil. In some cases, a single type of essential oil is used. In other cases, a mixture of different essential oils is used. In some embodiments, one or more essential oil is selected from chamomile, lavender, eucalyptus, peppermint, rosemary, clary sage, juniper, yarrow, clove, wintergreen, cayenne, ginger, sandalwood, marjoram, helichrysum, frankincence, vetiver, black pepper, lemongrass, rose geranium, bergamot, chenopodium, ylang-ylang, or L-menthol.

The additive may comprise a permeation enhancer. For example, any suitable permeation enhancer for topical and/or transdermal formulations may be used. In some cases, a single type of permeation enhancer is used. In other cases, the permeation enhancer may comprise a mix of permeation enhancers. For example, suitable permeation enhancers may include solvents (e.g., ethanol, acetone, polyethylene glycol, glycerol, propylene glycol), surfactants (Brij30® [polyoxyethylene(4)lauryl ether], Brij72® [polyoxyethylene (2) stearyl ether], Span 20 [sorbitan monolaurate]), azones (N-Acyl hexahydro-2-oxo-1H-azepines, N-Alkylmorpholine-2,3-diones), terpenes (e.g., limonene, carvone), fatty alcohols and fatty acids (e.g., lauryl alcohol, linolenyl alcohol, oleic acid and lauric acid) and/or others (e.g., lecithin, sodium de-oxy cholate). In some embodiments, the permeation enhancer comprises a surfactant. In some embodiments, the permeation enhancer is selected from sodium lauryl sulfate, polyoxyethylene-2-oleyl ether, or polyoxyethylene-2-stearyl ether. In some embodiments, the permeation enhancer comprises an oil. For example, the oil may be medium chain triglycerides (MCT) oil, coconut oil, or palm kernel oil. In some embodiments, the permeation enhancer comprises a fatty acid. For example, the fatty acid may be a triglyceride, one or more medium-chain triglycerides (MCTs), oleic acid, lauric acid, myristic acid, or capric acid. In some embodiments, the permeation enhancer comprises a glycol. For example, the permeation enhancer may be diethylene glycol or tetraethylene glycol. In some embodiments, the permeation enhancer comprises an alkyl group, and aryl group, and/or an azone. In some embodiments, one or more permeation enhancer is selected from ethanol, chenopodium, ylang-ylang, L-menthol, eucalyptus, sodium lauryl sulfate, polyoxyetylene-2-oleyl ether, polyoxyethylene-2-stearyl ether, oleic acid, lauric acid, myristic acid, capric acid, diethylene glycol, tetraethylene glycol, or cyclic urea.

The additive may comprise a formulation of components comprising a medicament described herein. For example, the formulation may comprise a modulator of the endocannabinoid system, such as a cannabinoid or combination of cannabinoids as described herein. In some embodiments, the formulation comprises a permeation enhancer as described herein. In some embodiments, the formulation comprises an essential oil as described herein. In some embodiments, the formulation comprises a terpene as described herein. In some embodiments, the formulation comprises a fatty acid amide hydrolase (FAAH) inhibitor. In some embodiments, the formulation comprises a cannabinoid and a fatty acid amide hydrolase (FAAH) inhibitor. In some embodiments, the formulation comprises two or more different cannabinoids and a fatty acid amide hydrolase (FAAH) inhibitor. In some embodiments, the formulation comprises an essential oil and a terpene. In some embodiments, the formulation comprises a permeation enhancer, an essential oil, and a terpene. In some embodiments, the formulation comprises a permeation enhancer, an essential oil, a terpene, and a FAAH inhibitor. In some embodiments, the formulation comprises a cannabinoid, an essential oil, and a terpene. In some embodiments, the formulation comprises a cannabinoid, a permeation enhancer, an essential oil, and a terpene. In some embodiments, the formulation comprises a cannabinoid, a permeation enhancer, an essential oil, a terpene, and a FAAH inhibitor. In some embodiments, the formulation comprises two or more different cannabinoids, an essential oil, and a terpene. In some embodiments, the formulation comprises two or more different cannabinoids, a permeation enhancer, an essential oil, and a terpene. In some embodiments, the formulation comprises two or more different cannabinoids, a permeation enhancer, an essential oil, a terpene, and a FAAH inhibitor. In some embodiments, the additive is formulated as a lotion or a balm.

The additive and/or medicament of the therapeutic bandage described herein may be formulated with or in a carrier for enhanced delivery to a user. In some cases, the carrier may be a vesicular carrier. For example, the vesicular carrier may comprise ethosomes, emulsomes, liposomes, enzymosomes, virosomes, pharmacosomes, transferosomes, sphingosomes, or niosomes. In some embodiments, the cannabinoid is formulated in the form of a vesicular carrier, for example ethosomes, emulsomes, liposomes, enzymosomes, virosomes, pharmacosomes, transferosomes, sphingosomes, or niosomes.

In some embodiments, the additive and/or medicament of the therapeutic bandage described herein is formulated to comprise an ethosomal carrier; a first cannabinoid and a second cannabinoid at a ratio of about 0.1:1.0, 0.2:1.0, 0.3:1.0, 0.4:1.0, 0.5:1.0, 0.6:1.0, 0.7:1.0, 0.8:1.0, 0.9:1.0, 1.0:1.0, 1.0:0.9, 1.0:0.8, 1.0:0.7, 1.0:0.6, 1.0:0.5, 1.0:0.4, 1.0:0.3, 1.0:0.2, or 1.0:0.1 by weight; limonene; and beta-myrcene. In other embodiments, the additive and/or medicament of the therapeutic bandage described herein is formulated to comprise an ethosomal carrier; a first cannabinoid and a second cannabinoid at a ratio of about 1:1; limonene; and beta-myrcene. In other embodiments, the additive and/or medicament of the therapeutic bandage described herein is formulated to comprise an ethosomal carrier, CBD and THC at a ratio of about 1:1 CBD:THC, limonene, and beta-myrcene. In other embodiments, the additive and/or medicament of the therapeutic bandage described herein is formulated to comprise an ethosomal carrier, a cannabinoid comprising CBD only, limonene, and beta-myrcene. In other embodiments, the additive and/or medicament of the therapeutic bandage described herein is formulated to comprise MCT oil; a first cannabinoid and a second cannabinoid at a ratio of about 0.1:1.0, 0.2:1.0, 0.3:1.0, 0.4:1.0, 0.5:1.0, 0.6:1.0, 0.7:1.0, 0.8:1.0, 0.9:1.0, 1.0:1.0, 1.0:0.9, 1.0:0.8, 1.0:0.7, 1.0:0.6, 1.0:0.5, 1.0:0.4, 1.0:0.3, 1.0:0.2, or 1.0:0.1 by weight; limonene; and beta-myrcene. In other embodiments, the additive and/or medicament of the therapeutic bandage described herein is formulated to comprise MCT oil; a first cannabinoid and a second cannabinoid at a ratio of about 1:1; limonene; and beta-myrcene. In other embodiments, the additive and/or medicament of the therapeutic bandage described herein is formulated to comprise MCT oil, CBD and THC at a ratio of about 1:1 CBD:THC, limonene, and beta-myrcene. In other embodiments, the additive and/or medicament of the therapeutic bandage described herein is formulated to comprise MCT oil, a cannabinoid comprising CBD only, limonene, and beta-myrcene. In other embodiments, the additive and/or medicament of the therapeutic bandage described herein is formulated to comprise one permeation enhancer or a combination of different permeation enhancers as described herein, CBD and THC at a ratio of about 10:1-1:10 CBD:THC, and one terpene or a combination of different terpenes as described herein. In other embodiments, the additive and/or medicament of the therapeutic bandage described herein is formulated to comprise one permeation enhancer or a combination of different permeation enhancers as described herein, one essential oil or a combination of different essential oils as described herein, CBD and THC at a ratio of about 10:1-1:10 CBD:THC, and one terpene or a combination of different terpenes as described herein. In other embodiments, the additive and/or medicament of the therapeutic bandage described herein is formulated to comprise one vesicular carrier or a combination of different vesicular carriers as described herein, CBD and THC at a ratio of about 10:1-1:10 CBD:THC, and one terpene or a combination of different terpenes as described herein.

In some embodiments, the additive of the therapeutic elastic bandage and/or kinesiology tape comprises one or more of Bedrocan®, Bedrobinol®, Bediol®, Bedica®, Cesamet®, Marinol®, or Sativex®.

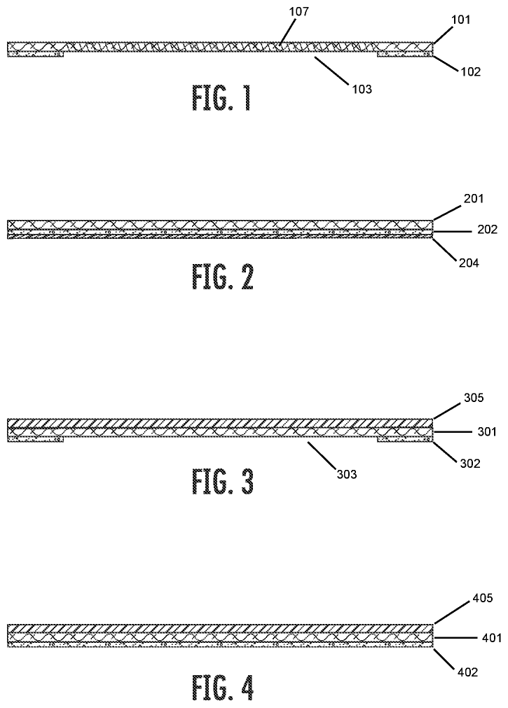

FIG. 1 shows a cross-sectional view of an embodiment of a kinesiology tape comprising a single layer of elastic material 101, an adhesive layer 102 that does not fully cover the skin-facing surface of the tape, and an additive 107 comprising a medicament to modulate the endocannabinoid system of a subject. In the embodiment shown in FIG. 1, the additive 107 is infused into the portion of the elastic material 103 where there is no adhesive layer beneath. In other embodiments, the additive may be infused into any part of the elastic material 101, including portions having an adhesive layer beneath. In yet other embodiments, instead of being infused into the elastic material, the additive may be provided as a component of the adhesive layer 102.

FIG. 2 shows a cross-sectional view of an embodiment of a kinesiology tape comprising a single layer of elastic material 201, an adhesive layer 202 that fully covers the skin-facing surface of the tape, and a removable protective sheet 204 on the adhesive layer. In other embodiments, a removable protective sheet 204 is present to protect an adhesive layer that does not fully cover the skin-facing surface of the tape (e.g., 102).

FIG. 3 shows a cross-sectional view of an embodiment of a kinesiology tape comprising a first elastic layer 301 and a second elastic layer 305, and an adhesive layer 302 that does not fully cover the skin-facing surface of the tape, thereby leaving a portion of elastic material 303 where there is no adhesive layer beneath. In some embodiments, an additive comprising a medicament to modulate the endocannabinoid system of a subject may be infused into a portion of the first layer 301 and/or the second layer 305.

FIG. 4 shows a cross-sectional view of an embodiment of a kinesiology tape comprising a first elastic layer 401 and a second elastic layer 405, and an adhesive layer 402 that fully covers the skin-facing surface of the tape. In some embodiments, an additive comprising a medicament to modulate the endocannabinoid system of a subject may be infused into a portion of the first layer 401 and/or the second layer 405.

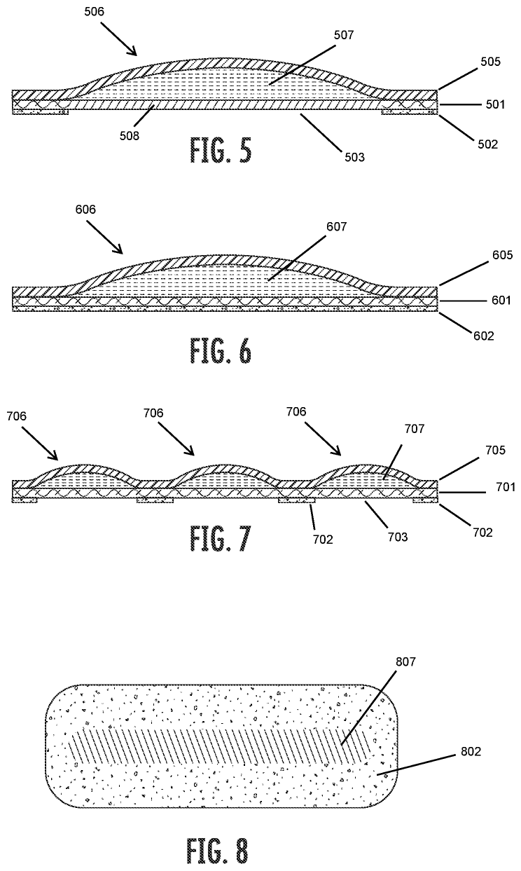

FIG. 5 shows a cross-sectional view of an embodiment of a kinesiology tape comprising a first elastic layer 501 and a second elastic layer 505, an adhesive layer 502 that does not fully cover the skin-facing surface of the tape 503, a reservoir 506 between the multiple layers, and an additive 507 comprising a medicament to modulate the endocannabinoid system of a subject concentrated in the reservoir. In the embodiment shown in FIG. 5, a different elastic material 508 is used underneath the reservoir 506 to facilitate diffusion of the medicament into a user's body. For example, the different elastic material 508 may comprise a porous membrane capable of controlled release of the medicament into the user.

FIG. 6 shows a cross-sectional view of an embodiment of a kinesiology tape comprising a first elastic layer 601 and a second elastic layer 605, an adhesive layer 602 that fully covers the skin-facing surface of the tape, a reservoir 606 between the multiple layers, and an additive 607 comprising a medicament to modulate the endocannabinoid system of a subject concentrated in the reservoir.

FIG. 7 shows a cross-sectional view of an embodiment of a kinesiology tape comprising a first elastic layer 701 and a second elastic layer 705, an adhesive layer 702 that does not fully cover the skin-facing surface of the tape 703, multiple reservoirs 706 between the multiple layers 701 and 705, and an additive 707 comprising a medicament to modulate the endocannabinoid system of a subject concentrated in the reservoir.

FIG. 8 shows a plan view of an embodiment of a kinesiology tape, wherein the locations of additive 807 and adhesive material 802 do not overlap.

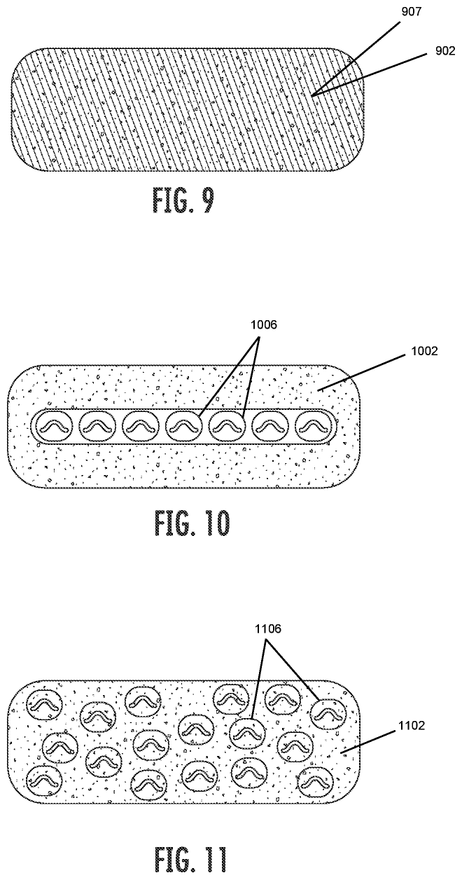

FIG. 9 shows a plan view of an embodiment of a kinesiology tape, wherein the locations of additive 907 and adhesive material 902 overlap.

FIG. 10 shows a plan view of an embodiment of a kinesiology tape, wherein the locations of reservoirs 1006 and adhesive material 1002 do not overlap.

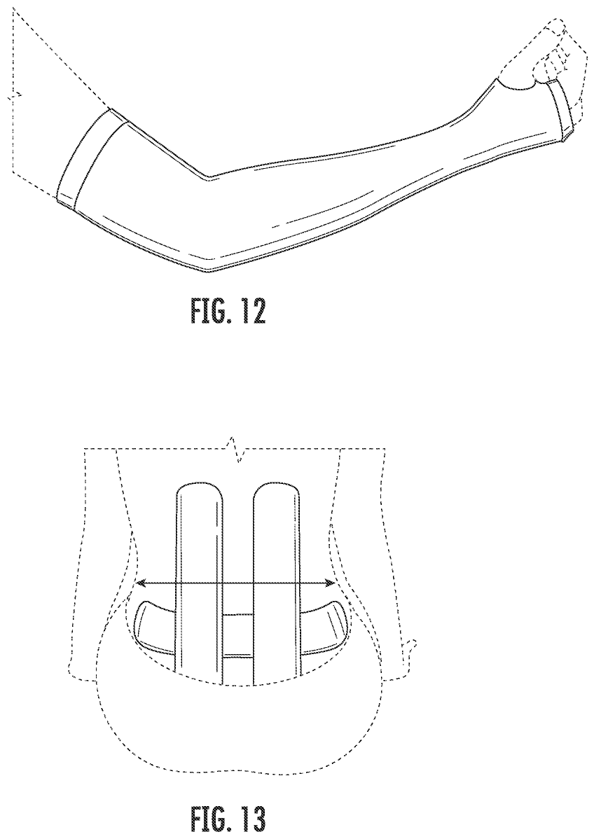

FIG. 11 shows a plan view of an embodiment of a kinesiology tape, wherein the locations of reservoirs 1106 and adhesive material 1102 overlap.

Any one of the aforementioned embodiments, for example as shown in FIGS. 1-11, may be modified to produce a therapeutic elastic bandage comprising no adhesive material on a surface of the bandage in contact with a user's skin. In some embodiments, such a therapeutic elastic bandage may be provided as a tube or a sleeve suitable to use for arms, legs, or any other suitable body part of a subject.

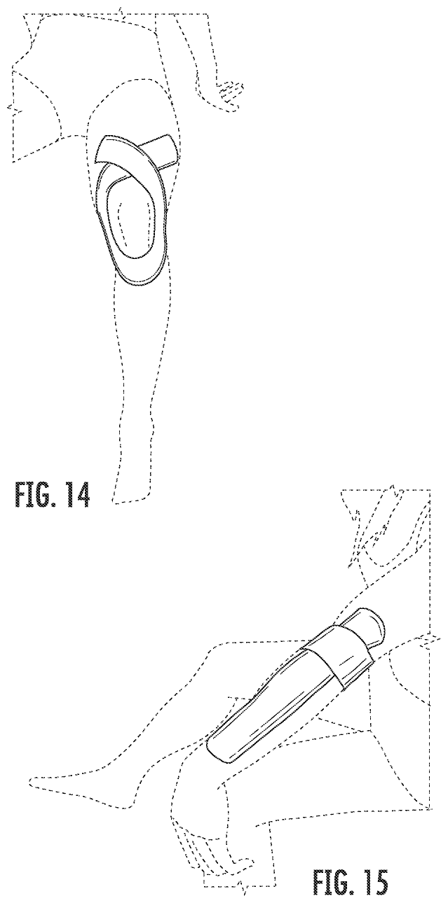

The invention provides a method for delivering an effective dose of a modulator of the endocannabinoid system to a subject by attaching or applying a therapeutic bandage comprising an elastic material and an additive to a portion of the body of the subject, wherein the additive comprises a medicament, and wherein the medicament comprises the modulator of the endocannabinoid system. In some embodiments, the invention provides a method for achieving a beneficial physiological effect in a subject by attaching or applying a therapeutic bandage comprising an elastic material and an additive to a portion of the body of the subject, wherein the additive comprises a medicament, and wherein the medicament comprises a modulator of the endocannabinoid system, thereby delivering an effective amount of the medicament to the subject. In some embodiments, the method is for modulating the endocannabinoid system of a subject, comprising attaching or applying the therapeutic bandage as described herein to a portion of the body of the subject. In some embodiments, the method is for achieving a beneficial effect in a disease, disorder or condition mediated by the endocannabinoid system, comprising attaching or applying the therapeutic bandage as described herein to a portion of the body of the subject. The subject may be a human subject or an animal subject. In some embodiments, the method comprises stretching the therapeutic elastic bandage and wrapping a portion of a body of a subject with the stretched bandage to compress an area. In some embodiments, the therapeutic elastic bandage is provided as a tube or a sleeve, and the method comprises pulling the bandage over a portion of a body of a subject to compress an area. For example, FIG. 12 shows an embodiment of a therapeutic elastic bandage positioned on the arm of a human subject. In other embodiments, the bandage shown in FIG. 12 may be modified, for example, by adapting its dimensions for other body parts, such as elbow, forearm, wrist, knee, ankle, etc. In some embodiments, the elastic material of the therapeutic bandage used in the method is a kinesiology tape as described herein. The kinesiology tape may be applied to lift skin away from underlying muscles and nerve endings to provide relief from pain. In some embodiments, the method comprises stretching the kinesiology tape and adhering the tape via its outer adhesive layer to a portion of the body of the subject. In other embodiments, the method comprises stretching a portion of the body of the subject and adhering unstretched or minimally stretched kinesiology tape via its outer adhesive layer to the stretched portion of the body of the subject. The kinesiology tape may be applied to skin on any part of the body, for example, Achilles tendon, ankle, lower back, upper back, calf, elbow, groin, hamstring, iliotibial band (IT band), knee, neck, plantar fasciitis, rib area, shin, shoulder, thigh, tow, and/or twist. Examples of a kinesiology tape attached to a portion of a body of a subject as described herein are shown in FIGS. 13-15. In some embodiments, the therapeutic elastic bandage and/or kinesiology tape is attached/applied to the skin of the subject for at least about 1 hour, at least about 3 hours, at least about 6 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 18 days, at least about 22 days, at least about 26 days, or at least about 30 days.

In some embodiments, the method comprising the attachment or application of the therapeutic elastic bandage and/or kinesiology tape to a subject as described herein promotes lymphatic drainage, improves muscle tone, provides pain relief, reduces swelling, reduces inflammation, reduces muscle spasms, reduces muscle cramps, reduces muscle fatigue, promotes recovery from bruises, promotes recovery from contusions, alleviates nausea, promotes muscle relaxation, provides muscle support, provides joint support, promotes muscle recovery, prevents muscle spasms/cramping, relieves muscle spasms/cramping, promotes muscle recovery, supports injured muscle without restricting range of movement, enhances strength and muscle tone, relieves pressure on pain receptors directly under the skin, facilitates blood circulation, facilitates lymphatic circulation, facilitates lymphatic drainage, drains swelling, facilitates removal of inflammatory cells away from an injured area quickly, relieves lymphedema, limits buildup of lactic acid, facilitates removal of lactic acid, delays onset of muscle soreness, and/or ameliorates post-exercise muscle soreness.

What is claimed is:

1. A therapeutic bandage comprising an elastic material and an additive, wherein the additive comprises a medicament, and wherein the medicament comprises a modulator of the endocannabinoid system, wherein the bandage is capable of delivering a biologically effective dose of the medicament to at least a portion of a body of a subject, and wherein the material comprises a front surface and a back surface, and wherein an adhesive material is applied to at least one surface.

2. The bandage of claim 1, wherein the elastic material comprises a kinesiology tape.

3. The bandage of claim 1, wherein the medicament comprises a cannabinoid or a fatty acid amide hydrolase (FAAH) inhibitor.

4. The bandage of claim 3, wherein the cannabinoid is a cannabidiol (CBD), a cannabichromene (CBC), a tetrahydrocannabinol (THC), a cannabigerol (CBG), or a combination thereof.

5. The bandage of claim 4, wherein the THC comprises delta-9-THC-acid and/or delta-8-THC, the CBD comprises CBD-acid, the CBC comprises CBC-acid, or wherein the CBG comprises CBG-acid.

6. The bandage of claim 3, wherein the medicament comprises CBD and THC.

7. The bandage of claim 6, wherein the medicament comprises a CBD/THC ratio of about 1:1 by weight.

8. The bandage of claim 1, wherein the additive comprises a terpene, an essential oil, a permeation enhancer, a cannabinoid, or combination thereof.

9. The bandage of claim 1, wherein (i) the terpene comprises an eucalyptol, borneol, alpha-bisabolo, camphene, delta-3-carene, humulene, linalool, beta-caryophyllene, myrcene, alpha-pinene, and limonene, (ii) wherein the essential oil comprises chamomile, lavender, eucalyptus, peppermint, rosemary, clary sage, juniper, yarrow, clove, wintergreen, cayenne, ginger, sandalwood, marjoram, helichrysum, frankincence, vetiver, black pepper, lemongrass, rose geranium, bergamot, chenopodium, ylang-ylang, and L-menthol, or (ii) wherein the permeation enhancer comprises a surfactant, an oil, a glycol, a fatty acid, an azone, a compound with an alkyl group, or a compound with an aryl group.

10. The bandage of claim 9, wherein (i) the surfactant is selected from the group consisting of sodium lauryl sulfate, polyoxyethylene-2-Oleyl ether, and polyoxyethylene-2-stearyl ether, (ii) wherein the permeation enhancer oil is selected from the group consisting of medium chain triglycerides (MCT) oil, coconut oil, and palm kernel oil, (iii) wherein the fatty acid is selected from the group consisting of a triglyceride or one or more medium-chain triglyceride, or (iv) wherein the glycol is selected from the group consisting of diethylene glycol and tetraethylene glycol.

11. The bandage of claim 1, wherein the additive comprises one or more permeation enhancer selected from the group consisting of ethanol, chenopodium, ylang-ylang, L-menthol, eucalyptus, sodium lauryl sulfate, polyoxyetylene-2-oleyl ether, polyoxyethylene-2-stearyl ether, medium chain triglycerides (MCT) oil, coconut oil, palm kernel oil, a triglyceride, one or more medium-chain triglycerides (MCTs), oleic acid, lauric acid, myristic acid, capric acid, diethylene glycol, tetraethylene glycol, and cyclic urea.

12. The bandage of claim 1, wherein the additive and/or medicament is formulated with or in a carrier for enhanced delivery to a user and the carrier is a vesicular carrier.

13. The bandage of claim 12, wherein the vesicular carrier is selected from the group consisting of ethosomes, emulsomes, liposomes, enzymosomes, virosomes, pharmacosomes, transferosomes, sphingosomes, and niosomes.

14. The bandage of claim 1, wherein the additive comprises (i) a formulation comprising an ethosomal carrier, CBD and THC at a ratio of about 1:1 CBD:THC, limonene, and beta-myrcene: (ii) a formulation comprising an ethosomal carrier, a cannabinoid comprising CBD only, limonene, and beta-myrcene; (iii) a formulation comprising MCT oil, CBD and THC at a ratio of about 1:1 CBD:THC, limonene, and beta-myrcene; (iv) a formulation comprising MCT oil, a cannabinoid comprising CBD only, limonene, and beta-myrcene; or (v) a formulation comprising a vesicular carrier, CBD and THC at a ratio of about 10:1-1:10 CBD:THC, and a terpene.

15. The bandage of claim 1, wherein the elastic material comprises one or more elastic layers.

16. The bandage of claim 15, wherein the one or more layers comprise microneedles.

17. The bandage of claim 15, wherein the one or more layers is abrasive.

18. The bandage of claim 1, wherein the adhesive is stretch-activated and/or heat-activated.

19. The bandage of claim 1, wherein the adhesive comprises an acrylic or poly-acrylic adhesive.

20. The bandage of claim 1, wherein the bandage comprises a reservoir.

21. The bandage of claim 20, wherein the reservoir comprises the additive.

22. The bandage of claim 1, wherein the additive is infused into a portion of one or more of the layers of elastic material.

23. A method for delivering an effective dose of a modulator of the endocannabinoid system to a portion of a body of a subject, comprising attaching or applying a therapeutic bandage to the skin of the subject, wherein the therapeutic bandage comprises an elastic material and an additive, wherein the additive comprises a medicament, and wherein the medicament comprises the modulator of the endocannabinoid system.
